(19) United States
(12) Patent Application Publication
(10) Pub. No.: US 2023/0092844 A1
(43) Pub. Date: Mar. 23, 2023

(54) PROCESS FOR MAKING SOLID METHYLGLYCINE DIACETATE ALKALI METAL SALTS

(71) Applicant: BASF SE, Ludwigshafen (DE)

(72) Inventors: Matthias VOGES, Ludwigshafen (DE); Lothar SEIDEMANN, Ludwigshafen (DE); Anika RITTER, Ludwigshafen (DE); Astrid SCHMIDT, Ludwigshafen (DE); Markus HARTMANN, Ludwigshafen (DE); Hartmut KOENIG, Ludwigshafen (DE); Frank JAEKEL, Ludwigshafen (DE); Rou Hua CHUA, Ludwigshafen (DE); Michael Klemens MUELLER, Ludwigshafen (DE)

(21) Appl. No.: 17/800,758

(22) PCT Filed: Feb. 11, 2021
(86) PCT No.: PCT/EP2021/053369

§ 371 Date: Aug. 18, 2022

(30) Foreign Application Priority Data
Feb. 20, 2020 (EP) 20158430.7

(51) Int. Cl. C07C 227/42

(52) U.S. Cl. CPC C07C 227/42

(57) ABSTRACT

Process for making solid methylglycine diacetate (MGDA) alkali metal salt (A), said process comprising the steps of
(a) providing a 40 to 60% by weight aqueous solution of said salt (A) having a temperature in the range of from 50 to 90° C.,
(b) crystallizing salt (A),
(c) removing said crystalline salt (A) from the mother liquor by filtration, and
(d) subjecting the water containing filter cake from step (c) to an extrusion step.

The present invention is directed towards a process for making solid methylglycine diacetate (MGDA) alkali metal salt (A), said process comprising the steps of
(a) providing a 40 to 60% by weight aqueous solution of said salt (A) having a temperature in the range of from 50 to 90° C.,
(b) crystallizing salt (A),
(c) removing said crystalline salt (A) from the mother liquor by filtration, and
(d) subjecting the water containing filter cake from step (c) to an extrusion step.

Furthermore, the present invention is directed towards certain column-shaped particles of methylglycine diacetate (MGDA) alkali metal salt (a).

Chelating agents of the aminocarboxylate type such as methyl glycine diacetic acid (MGDA) and glutamic acid diacetic acid (GLDA) and their respective alkali metal salts are useful sequestrants for alkaline earth metal ions such as Ca²⁺ and Mg²⁺. A lot of aminocarboxylates show good biodegradability and are thus environmentally friendly. For that reason, they are recommended and used for various purposes such as laundry detergents and for automatic dishwashing (ADW) formulations, in particular for so-called phosphate-free laundry detergents and phosphate-free ADW formulations.

Depending on the type of product—liquid home care and fabric care products versus solid home care and fabric care products—and the manufacturing process of solid home care and fabric care products care product manufacturers may either prefer to handle solutions of aminocarboxylates or solid arminocarboxylates, for example joint spray drying or solid mixing. Powders and granules of aminocarboxylates may be shipped economically due to their high active ingredient content that goes along with low water content. Therefore, convenient processes for providing granules are still of great commercial interest.

In EP 0 845 846 A, a process to make crystalline salts of MGDA is disclosed. A water-containing super-cooled melt of MGDA is seeded with MGDA powder. The process has its shortcomings, though, because it is tedious to remove the crystals from the crystallization vessel. In WO 2018/153876, another process to crystallize MGDA and related compounds is disclosed.

In WO 2009/103822, a process is disclosed in which slurries are granulated that have a certain solids content, with a gas inlet temperature of 120° C. or less. In WO 2012/168739, a process is disclosed wherein slurries of complexing agents are spray-dried under non-agglomerating conditions.

Both processes have their shortcomings. A low gas inlet temperature requires highly concentrated slurries or a huge amount of gas per unit of granule. A process using non-agglomerating conditions provides for powders only.

It is desired to provide chelating agents in solid form that are less hygroscopic and give no or little raise to yellowing upon contact with percarbonate. It is therefore an objective of the present invention to provide chelating agents in solid form that are less hygroscopic and give no or little raise to yellowing upon contact with percarbonate, and it is an objective of the present invention to provide a process for manufacturing such chelating agents in solid form.

Accordingly, the process defined at the outset has been found, hereinafter also referred to as inventive process or as process according to the present invention. The inventive process comprises several steps that may be referred to as step (a), step (b) etc. and that will be explained in more detail below.

In step (a), an aqueous solution of methylglycine diacetate (MGDA) alkali metal salt (A) is provided, hereinafter also referred to as “salt (A)”. In this context, alkali metal salts are selected from lithium salts, sodium salts, potassium salts, rubidium salts, and cesium salts and combinations of at least two of the foregoing, with potassium salts being preferred and sodium salts being more preferred.

Salts (A) may refer to fully neutralized MGDA and to partially neutralized MGDA.

In one embodiment of the present invention, salt (A) is selected from compounds according to general formula (I)

[CH₃—CH(COO)—N(CH₂—COO)₂]M₃₋ₓHₓ (I)

wherein

M is selected from alkali metal cations, same or different, preferably K or Na or combinations thereof, and even more preferably Na, and

x is in the range of from zero to 1.0, preferably zero to 0.5.

In any way, aqueous solutions of salt (A) may bear a cation other than alkali metal. It is thus possible that minor amounts, such as 0.01 to 5 mol-% of total MGDA, respectively, bear alkali earth metal cations such as Mg²⁺ or Ca²⁺, or an Fe²⁺ or Fe³⁺ cation.

As mentioned above, in step (a) an aqueous solution of salt (A) is provided. Aqueous solutions are defined herein as solutions with no solid particles detectable by visual inspection. Aqueous solutions may contain minor amounts of organic solvent that is or are miscible with water, for example ethanol, 1,2-propylenglycol, ethylene glycol, for example in a volume ratio of water:organic solvent 5:1 to 100:1. Preferably, however, aqueous solutions provided in step (a) do not contain detectable amounts of organic solvent.

Salts (A) are selected from the racemic mixtures, the D-isomers and the L-isomers, and from mixtures of the D- and L-isomers other than the racemic mixtures. Preferably, salt (A) is selected from the racemic mixture and from mixtures containing in the range of from 55 to 95 mole-% of the L-isomer, the balance being D-isomer. Particularly preferred are solutions of salt (A) being selected from the racemic mixture and mixtures of the enantiomers with predominantly the L-enantiomer with an ee value in the range of from 0.1 to 30%. Other particularly preferred embodiments are racemic mixtures.

In one embodiment of the present invention, aqueous solutions of salt (A) may contain one or more impurities that may result from the synthesis of the respective salt (A). Such impurities may be selected from propionic acid, lactic acid, alanine, nitrilotriacetic acid (NTA) or the like and their respective alkali metal salts. Such impurities are usually present in minor amounts. “Minor amounts” in this context refer to a total of 0.1 to 5% by weight, referring to salt (A), preferably up to 2.5% by weight. In the context of the present invention, such minor amounts are neglected when determining the composition of granule made according to the inventive process.

Aqueous solutions are defined as transparent solutions with no solid particles detectable by visual inspection.

In one embodiment of the present invention, aqueous solutions of salt (A) contain one or more inorganic salts, for example alkali metal hydroxide, alkali metal (bi)carbonate, alkali metal formate, or the like, for example in amounts of 0.5 to 10% by weight, referring to salt (A).

(Co)polymer (B) is selected from polymers (B) of (meth)acrylic acid and of copolymers (B) of (meth)acrylic acid, preferably of acrylic acid, partially or fully neutralized with alkali. In the context of the present invention, copolymers (B) are those in which at least 50 mol-% of the comonomers are (meth)acrylic acid, preferably at least 75 mol-%, even more preferably 80 to 99 mol-%.

Suitable comonomers for copolymers (B) are ethylenically unsaturated compounds, such as styrene, isobutene, ethylene, α-olefins such as propylene, 1-butylene, 1-hexene, and ethylenically unsaturated dicarboxylic acids and their alkali metal salty and anhydrides such as but not limited to maleic acid, fumaric acid, itaconic acid disodium maleate, disodium fumarate, itaconic anhydride, and especially maleic anhydride. Further examples of suitable comonomers are C₁-C₄-alkyl esters of (meth)acrylic acid, for example methyl acrylate, methyl methacrylate, ethyl acrylate, ethyl methacrylate, n-butyl acrylate.

In one embodiment of the present invention, (co)polymer (B) is selected from copolymers of (meth)acrylic acid and a comonomer bearing at least one sulfonic acid group per molecule. Comonomers bearing at least one sulfonic acid group per molecule may be incorporated into copolymer (B) as free acid or least partially neutralized with alkali. Particularly preferred sulfonic-acid-group-containing comonomers are 1-acrylamido-1-propanesulfonic acid, 2-acrylamido-2-propanesulfonic acid, 2-acrylamido-2-methylpropanesulfonic acid (AMPS), 2-methacrylamido-2-methylpropanesulfonic acid, 3-methacrylamido-2-hydroxypropanesulfonic acid, allylsulfonic acid, methallylsulfonic acid, allyloxybenzenesulfonic acid, methallyloxybenzenesulfonic acid, 2-hydroxy-3-(2-propenyloxy)propanesulfonic acid, 2-methyl-2-propene-1-sulfonic acid, styrenesulfonic acid, vinylsulfonic acid, 3-sulfopropyl acrylate, 2-sulfoethyl methacrylate, 3-sulfopropyl methacrylate, sulfomethacrylamide, sulfomethylmethacrylamide, and salts of said acids, such as the sodium salts, potassium salts or ammonium salts thereof.

Copolymers (B) may be selected from random copolymers, alternating copolymers, block copolymers and graft copolymers, alternating copolymers and especially random copolymers being preferred.

Useful copolymers (B) are, for example, random copolymers of acrylic acid and methacrylic acid, random copolymers of acrylic acid and maleic anhydride, ternary random copolymers of acrylic acid, methacrylic acid and maleic anhydride, random or block copolymers of acrylic acid and styrene, random copolymers of acrylic acid and methyl acrylate. More preferred are homopolymers of methacrylic acid. Even more preferred are homopolymers of acrylic acid.

(Co)polymer (B) may constitute straight-chain or branched molecules. Branching in this context will be when at least one repeating unit of such (co)polymer (B) is not part of the main chain but forms a branch or part of a branch. Preferably, (co)polymer (B) is not cross-linked.

In one embodiment of the present invention, (co)polymer (B) has an average molecular weight M_{W} in the range of from 1,200 to 30,000 g/mol, preferably from 2,500 to 15,000 g/mol and even more preferably from 3,000 to 10,000 g/mol, determined by gel permeation chromatography (GPC) and referring to the respective free acid.

Preferably, however, aqueous solutions of salt (A) as provided in step (a) does not contain any (co)polymers of (meth)acrylic acid.

In one embodiment of the present invention, the aqueous solution provided in step (a) has a concentration of salt (A) In the range of from 35 to 60% by weight, preferably 40 to 50% by weight and even more preferably 45 to 50% by weight.

In one embodiment of the present invention, the aqueous solution provided in step (a) has a temperature in the range of from 50 to 95° C., preferably 60 to 80° C.

In one embodiment of the present invention, such aqueous solution according to step (a) has a pH value in the range of from 8 to 14, preferably from 9 to 13.5 and even more preferably at least 9.5. The pH value is determined at ambient temperature.

Solutions according to step (a) may be obtained by various methods. It is possible, e.g., to heat a given aqueous solution of component (a) to 50 to 95° C., or to dissolve salt (A) in water under heating and, if applicable, removal of some of the water, for example by evaporation. In an alternative embodiment, it is possible to start the inventive process with a solution of salt (A) that is stemming directly from the synthesis, in particular the saponification step, and to incompletely cool down such solution.

In step (b), salt (A) is crystallized, for example by evaporation crystallization.

In one embodiment of the present invention, step (b) is preferably initiated by seeding, where seeding material is of 85 to 99% by weight orthorhombic alkali metal salt of MGDA, referring to the entire content of the respective salt. The amount of seed is preferably 0.5 to 2% by weight of the total amount of salt (A) in the slurry. From the slurry so obtained water may be removed within one to seven hours, preferably in two to five hours and even more preferably in three to four hours.

From step (b), a slurry of crystalline salt (A) is obtained.

In step (c), said crystalline salt (A) is removed from the mother liquor by a solid-liquid separation step, preferably by filtration. Said filtration may be batch-wise, especially in laboratory-scale experiments, or continuous, especially in ton-scale or multi-ton scale. Examples of suitable batch filtration methods are pressure filtration by filter presses, diaphragm filter presses and pressure nutsche filters as well as centrifugal filtration by peeler or inverting filter centrifuges. Examples of suitable continuous filtrations are vacuum belt filters and rotary pressure filters. An example of a semi-continuous filtration is an automatic tower pressure filter with endless filter cloth and diaphragm squeeze device. A water-containing filter cake is obtained that is transferred to step (d).

In step (c), an aqueous filtrate is obtained as well. It is possible to recycle said aqueous filtrate.

The water-containing filter cake from step (c) may hereinafter also be referred to as “wet filter cake” or “wet filter cake from step (c)”.

In one embodiment of the present invention, the wet filter cake has a water content in the range of from 1 to 40% by weight, referring to the entire wet filter cake and determined by removal of water at 80° C. in vacuo.

In one embodiment of the present invention, the water content of the wet filter cake is reduced by treatment with a gas like air or nitrogen or nitrogen-enriched air, for example by blowing said gas through the wet filter cake.

In one embodiment of the present invention, step (c) includes a diaphragm squeeze subsequently to filtration. By said diaphragm squeeze, the water content of the wet filter cake is reduced by squeezing the filter cake with a water or gas inflated diaphragm in the chamber of a filter press or in an automatic tower pressure filter. The diaphragm squeeze pressure is preferably in the range of from 1 to 30 bar.

In one embodiment of the present invention, step (c) includes washing the wet filter cake, for example with water, with aqueous alkali metal hydroxide solution or with methanol. Preferably, the wet filter cake from step (c) is washed with water, with aqueous alkali metal hydroxide solution or with methanol each with a temperature in the range of from 2 to 15° C., preferably 5 to 10° C.

In step (d), the water-containing filter cake from step (c) is subjected to an extrusion step. As a result of the extrusion step, the water content is reduced, for example to a residual moisture content in the range of from 1 to 20% by weight, preferably 2 to 5% by weight.

In one embodiment of the present invention, step (d) is carried out in a piston press, a twin screw extruder or a single-screw extruder, single screw extruders being preferred.

In one embodiment of the present invention, the diameter of a single-screw screw in an extruder is in the range of from 10 to 100 cm, preferably, from 15 cm to 50 cm. The length of the single-screw extruder may be in the range of from 20 to 300 cm, preferably from 50 cm to 200 cm.

In one embodiment of the present invention, the temperature during step (d) is in the range of from 40 to 80° C., preferred are 65 to 75° C.

In one embodiment of the present invention, the pressure at the exit of the respective piston press, twin screw extruder or single-screw extruder is in the range of from 0.1 to 200 bar.

In one embodiment of the present invention, the residence time of the filter cake in the extruder is from 5 seconds to 1000 seconds, preferably from 30 to and 300 seconds.

In one embodiment of the present innovation, the filter cake is pressed through a die of holes at the outlet of the extruder. Diameter of the holes defines the diameter of the obtained strands of compressed filter cake, which is between 0.1 mm and 20 mm, preferably, between 0.5 and 2.0 mm.

In one embodiment of the present innovation, the length of the obtained strands of compressed filter cake is defined by a cutting device, which is between 1 mm and 50 mm, preferably, between 1 and 20 mm. The cutting device can be a rotating knife, which is fixed at the outlet of the extruder.

In one embodiment of the present invention, at the beginning of step (d), some powder of salt (A) is added to the wet filter cake, for example 1 to 15% by weight, referring to the wet filter cake.

By performing step (d), an extrudate is obtained. Said extrudate has advantageous properties, e.g., low tendency of yellowing and very low hygroscopicity.

In one embodiment of the present invention, step (d) is followed by at least one step selected from
(e) drying the extrudate,
(f) grinding the extrudate.

For grinding the extrudate, step (e), at least one the following measures may be taken:

For step (e), the extrudate may be dried at a temperature in the range of from 60 to 80° C.

By performing steps (e) and (f), an extrudate in the form of columns is formed, hereinafter also referred to as inventive extrudate. Inventive extrudates show excellent properties with respect to low tendency of yellowing and very low hygroscopicity.

A further aspect of the present invention is directed towards particles of salts (A) in the shape of columns, hereinafter also referred to as inventive particles of salt (A). Such inventive particles of salt (A) have an average diameter in the range of from 0.1 to 20 mm and a length of at least 1 mm, said particles containing
(A1) in the range of from 1 to 10% by weight monoclinic alkali metal salt of MGDA,
(A2) in the range of from 90 to 99% by weight orthorhombic alkali metal salt of MGDA,

percentages referring to the entire content of the crystalline salt (A), and wherein the crystalline content is 80 to 99% by weight referring to the entire content of the respective salt (A).

Inventive salts show extremely low hygroscopicity, and they may be obtained in accordance with the inventive process.

The percentages of (A1) and (A2) as well as the percentage of crystalline versus amorphous salt (A) are determined by X-ray diffraction.

In one embodiment of the present invention, inventive particles of salts (A) have a particle size (diameter) in the range of from 10 μm to 5 mm, preferably 100 μm to 1.5 mm.

In one embodiment of the present invention, in inventive particles of salts (A) said salt (A) is selected from compounds according to general formula (I)

[CH₃—CH(COO)—N(CH₂—COO)₂]M₃₋ₓHₓ (I)

wherein M is selected from alkali metal cations, same or different, see above, and x is in the range of from zero to 1.0.

In a preferred embodiment of the present invention, in inventive particles of salts (A) said salt (A) is the trisodium salt.

In one embodiment of the present invention, inventive particles of salts (A) have a bulk density in the range of from 400 g/L to 1200 g/l, preferably 700 to 1000 g/l. The bulk density is measured according to DIN ISO 697.

Inventive particles of salts (A) are excellently suited for the manufacture of laundry detergents or cleaners.

Another aspect of the present invention is therefore the use of inventive particles of salts (A) for the manufacture of a cleaning agent that may contain at least one bleaching agent, and in particular for the manufacture of cleaning agent for fibers or hard surfaces, wherein said cleaning agent contains at least one peroxy compound. Another aspect of the present invention is a process for making at a cleaning agent by combining inventive particles of salts (A) with at least one bleaching agent, preferably at least one peroxy compound. Another aspect of the present invention is a cleaning agent, hereinafter also being referred to as inventive cleaning agent.

Inventive cleaning agents may contain at least one bleaching agent and inventive particles of salts (A). Inventive cleaning agents show a reduced tendency for yellowing and therefore have an extended shelf-life.

Examples of suitable peroxy compounds are sodium perborate, anhydrous or for example as monohydrate or as tetrahydrate or so-called dihydrate, sodium percarbonate, anhydrous or, for example, as monohydrate, hydrogen peroxide, persulfates, organic peracids such as peroxylauric acid, peroxystearic acid, peroxy-α-naphthoic acid, 1,12-diperoxydodecanedioic acid, perbenzoic acid, peroxylauric acid, 1,9-diperoxyazelaic acid, diperoxyisophthalic acid, in each case as free acid or as alkali metal salt, in particular as sodium salt, also sulfonylperoxy acids and cationic peroxy acids.

In a preferred embodiment, peroxy compound is selected from inorganic percarbonates, persulfates and perborates. Examples of sodium percarbonates are 2 Na₂CO₃. 3 H₂O₂. Examples of sodium perborate are (Na₂[B(OH)₂(O₂)]₂), sometimes written as NaBO₂. O₂. 3H₂O instead. Most preferred peroxy compound is sodium percarbonate.

The term “cleaning agents” includes compositions for dishwashing, especially hand dishwash and automatic dishwashing and ware-washing, and compositions for hard surface cleaning such as, but not limited to compositions for bathroom cleaning, kitchen cleaning, floor cleaning, de-scaling of pipes, window cleaning, car cleaning including truck cleaning, furthermore, open plant cleaning, cleaning-in-place, metal cleaning, disinfectant cleaning, farm cleaning, high pressure cleaning, and in addition, laundry detergent compositions.

Such cleaning agents may be liquids, gels or preferably solids at ambient temperature, solids cleaning agents being preferred. They may be in the form of a powder or in the form of a unit dose, for example as a tablet.

In one embodiment of the present invention, inventive cleaning agents may contain in the range of from 2 to 50% by weight of inventive particles of salts (A), in the range of from 0.5 to 15% by weight of bleach.

Percentages are based on the solids content of the respective inventive cleaning agent.

Inventive cleaning agents may contain further ingredients such as one or more surfactants that may be selected from non-ionic, zwitterionic, cationic, and anionic surfactants. Other ingredients that may be contained in inventive cleaning agents may be selected from bleach activators, bleach catalysts, corrosion inhibitors, sequestering agents other than chelating agent (A), enzymes, fragrances, dyestuffs, antifoams, and builders.

Particularly advantageous inventive cleaning agents may contain one or more complexing agents other than MGDA or GLDA. Advantageous detergent compositions for cleaners and advantageous laundry detergent compositions may contain one or more sequestrant (chelating agent) other than a mixture according to the present invention. Examples for sequestrants other than a mixture according to the present invention are IDS (iminodisuccinate), citrate, phosphonic acid derivatives, for example the disodium salt of hydroxyethane-1,1-diphosphonic acid (“HEDP”), and polymers with complexing groups like, for example, polyethyleneimine in which 20 to 90 mole-% of the N-atoms bear at least one CH₂COO⁻ group, and their respective alkali metal salts, especially their sodium salts, for example IDS-Na₄, and trisodium citrate, and phosphates such as STPP (sodium tripolyphosphate). Due to the fact that phosphates raise environmental concerns, it is preferred that advantageous inventive cleaning agents are free from phosphate. “Free from phosphate” should be understood in the context of the present invention, as meaning that the content of phosphate and polyphosphate is in sum in the range from 10 ppm to 0.2% by weight, determined by gravimetric methods and referring to the respective inventive cleaning agent.

Inventive cleaning agents may contain one or more surfactant, preferably one or more non-ionic surfactant.

Preferred non-ionic surfactants are alkoxylated alcohols, di- and multiblock copolymers of ethylene oxide and propylene oxide and reaction products of sorbitan with ethylene oxide or propylene oxide, alkyl polyglycosides (APG), hydroxyalkyl mixed ethers and amine oxides.

Preferred examples of alkoxylated alcohols and alkoxylated fatty alcohols are, for example, compounds of the general formula (II)

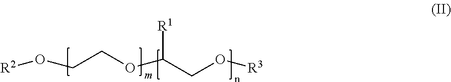

in which the variables are defined as follows:

R¹ is identical or different and selected from hydrogen and linear C₁-C₁₀-alkyl, preferably in each case identical and ethyl and particularly preferably hydrogen or methyl,

R² is selected from C₈-C₂₂-alkyl, branched or linear, for example n-C₈H₁₇, n-C₁₀H₂₁, n-C₁₂H₂₅, n-C₁₄H₂₉, n-C₁₆H₃₃ or n-C₁₈H₃₇,

R³ is selected from C₁-C₁₀-alkyl, methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl or isodecyl,

m and n are in the range from zero to 300, where the sum of n and m is at least one, preferably in the range of from 3 to 50. Preferably, m is in the range from 1 to 100 and n is in the range from 0 to 30.

In one embodiment, compounds of the general formula (II) may be block copolymers or random copolymers, preference being given to block copolymers.

Other preferred examples of alkoxylated alcohols are, for example, compounds of the general formula (III)

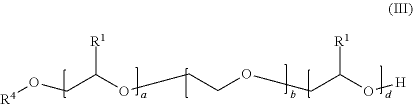

in which the variables are defined as follows:

R¹ is identical or different and selected from hydrogen and linear C₁-C₀-alkyl, preferably identical in each case and ethyl and particularly preferably hydrogen or methyl,

R⁴ is selected from C₆-C₂₀-alkyl, branched or linear, in particular n-C₈H₁₇, n-C₁₀H₂₁, n-C₁₂H₂₅, n-C₁₄H₂₉, n-C₁₆H₃₃, n-C₁₈H₃₇,

a is a number in the range from zero to 10, preferably from 1 to 6,

b is a number in the range from 1 to 80, preferably from 4 to 20,

d is a number in the range from zero to 50, preferably 4 to 25.

The sum a+b+d is preferably in the range of from 5 to 100, even more preferably in the range of from 9 to 50.

Preferred examples for hydroxyalkyl mixed ethers are compounds of the general formula (IV)

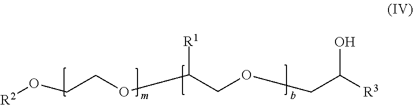

in which the variables are defined as follows:

R¹ is identical or different and selected from hydrogen and linear C₁-C₁₀-alkyl, preferably in each case identical and ethyl and particularly preferably hydrogen or methyl,

R² is selected from C₈-C₂₂-alkyl, branched or linear, for example iso-C₁₁H₂₃, iso-C₁₃H₂₇, n-C₈H₁₇, n-C₁₀H₂₁, n-C₁₂H₂₅, n-C₁₄H₂₉, n-C₁₆H₃₃ or n-C₁₈H₃₇,

R³ is selected from C₁-C₁₈-alkyl, methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl, isodecyl, n-dodecyl, n-tetradecyl, n-hexadecyl, and n-octadecyl.

The variables m and n are in the range from zero to 300, where the sum of n and m is at least one, preferably in the range of from 5 to 50. Preferably, m is in the range from 1 to 100 and n is in the range from 0 to 30.

Compounds of the general formula (II) and (III) may be block copolymers or random copolymers, preference being given to block copolymers.

Further suitable nonionic surfactants are selected from di- and multiblock copolymers, composed of ethylene oxide and propylene oxide. Further suitable nonionic surfactants are selected from ethoxylated or propoxylated sorbitan esters. Amine oxides or alkyl polyglycosides, especially linear C₄-C₁₆-alkyl polyglucosides and branched C₈-C₁₄-alkyl polyglycosides such as compounds of general average formula (V) are likewise suitable.

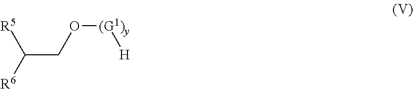

wherein the variables are defined as follows:

R⁵ is C₁-C₄-alkyl, in particular ethyl, n-propyl or isopropyl,

R⁶ is —(CH₂)₂—R⁵,

G¹ is selected from monosaccharides with 4 to 6 carbon atoms, especially from glucose and xylose,

y in the range of from 1.1 to 4, y being an average number.

An overview of suitable further nonionic surfactants can be found in EP-A 0 851 023 and in DE-A 198 19 187.

Mixtures of two or more different nonionic surfactants may also be present.

Other surfactants that may be present are selected from amphoteric (zwitterionic) surfactants and anionic surfactants and mixtures thereof.

Examples of amphoteric surfactants are those that bear a positive and a negative charge in the same molecule under use conditions. Preferred examples of amphoteric surfactants are so-called betaine-surfactants. Many examples of betaine-surfactants bear one quaternized nitrogen atom and one carboxylic acid group per molecule. A particularly preferred example of amphoteric surfactants is cocamidopropyl betaine (lauramidopropyl betaine).

Examples of amine oxide surfactants are compounds of the general formula (VI)

R⁷R⁸R⁹N->O (VI)

wherein R⁷, R⁸ and R⁹ are selected independently from each other from aliphatic, cycloaliphatic or C₂-C₄-alkylene C₁₀-C₂₀-alkylamido moieties. Preferably, R⁷ is selected from C₈-C₂₀-alkyl or C₂-C₄-alkylene C₁₀-C₂₀-alkylamido and R⁸ and R⁹ are both methyl.

A particularly preferred example is lauryl dimethyl aminoxide, sometimes also called lauramine oxide. A further particularly preferred example is cocamidylpropyl dimethylaminoxide, sometimes also called cocamidopropylamine oxide.

Examples of suitable anionic surfactants are alkali metal and ammonium salts of C₈-C₁₈-alkyl sulfates, of C₈-C₁₈-fatty alcohol polyether sulfates, of sulfuric acid half-esters of ethoxylated C₄-C₁₂-alkylphenols (ethoxylation: 1 to 50 mol of ethylene oxide/mol), C₁₂-C₁₈ sulfo fatty acid alkyl esters, for example of C₁₂-C₁₈ sulfo fatty acid methyl esters, furthermore of C₁₂-C₁₈-alkylsulfonic acids and of C₁₀-C₁₈-alkylarylsulfonic acids. Preference is given to the alkali metal salts of the aforementioned compounds, particularly preferably the sodium salts.

Further examples for suitable anionic surfactants are soaps, for example the sodium or potassium salts of stearoic acid, oleic acid, palmitic acid, ether carboxylates, and alkylether phosphates.

Preferably, laundry detergent compositions contain at least one anionic surfactant.

In one embodiment of the present invention, inventive cleaning agents that are determined to be used as laundry detergent compositions may contain 0.1 to 60% by weight of at least one surfactant, selected from anionic surfactants, amphoteric surfactants and amine oxide surfactants.

In one embodiment of the present invention, inventive cleaning agents that are determined to be used for hard surface cleaning may contain 0.1 to 60% by weight of at least one surfactant, selected from anionic surfactants, amphoteric surfactants and amine oxide surfactants.

In a preferred embodiment, inventive cleaning agents do not contain any anionic detergent.

Inventive cleaning agents may comprise one or more bleach catalysts. Bleach catalysts can be selected from bleach-boosting transition metal salts or transition metal complexes such as, for example, manganese-, iron-, cobalt-, ruthenium- or molybdenum-salen complexes or carbonyl complexes. Manganese, iron, cobalt, ruthenium, molybdenum, titanium, vanadium and copper complexes with nitrogen-containing tripod ligands and also cobalt-, iron-, copper- and ruthenium-amine complexes can also be used as bleach catalysts.

Inventive cleaning agents may comprise one or more bleach activators, for example N-methylmorpholinium-acetonitrile salts (“MMA salts”), trimethylammonium acetonitrile salts, N-acylimides such as, for example, N-nonanoylsuccinimide, 1,5-diacetyl-2,2-dioxohexahydro-1,3,5-triazine (“DADHT”) or nitrile quats (trimethylammonium acetonitrile salts).

Further examples of suitable bleach activators are tetraacetylethylenediamine (TAED) and tetraacetylhexylenediamine.

Inventive cleaning agents may comprise one or more corrosion inhibitors. In the present case, this is to be understood as including those compounds which inhibit the corrosion of metal. Examples of suitable corrosion inhibitors are triazoles, in particular benzotriazoles, bisbenzotriazoles, aminotriazoles, alkylaminotriazoles, also phenol derivatives such as, for example, hydroquinone, pyrocatechol, hydroxyhydroquinone, gallic acid, phloroglucinol or pyrogallol.

In one embodiment of the present invention, inventive cleaning agents comprise in total in the range from 0.1 to 1.5% by weight of corrosion inhibitor.

Inventive cleaning agents may comprise one or more builders, selected from organic and inorganic builders. Examples of suitable inorganic builders are sodium sulfate or sodium carbonate or silicates, in particular sodium disilicate and sodium metasilicate, zeolites, sheet silicates, in particular those of the formula α-Na₂Si₂O₅, β-Na₂Si₂O₅, and δ-Na₂Si₂O₅, also fatty acid sulfonates, α-hydroxypropionic acid, alkali metal malonates, fatty acid sulfonates, alkyl and alkenyl disuccinates, tartaric acid diacetate, tartaric acid monoacetate, oxidized starch, and polymeric builders, for example polycarboxylates and polyaspartic acid.

Examples of organic builders are especially polymers and copolymers other such as (co)polymers (B) and include polymers and copolymers than (co)polymer (B), or one additional (co)polymer (B). In one embodiment of the present invention, organic builders are selected from polycarboxylates, for example alkali metal salts of (meth)acrylic acid homopolymers or (meth)acrylic acid copolymers, partially or completely neutralized with alkali.

Suitable comonomers for (meth)acrylic acid are monoethylenically unsaturated dicarboxylic acids such as maleic acid, fumaric acid, maleic anhydride, itaconic acid and citraconic acid. A suitable polymer is in particular polyacrylic acid, which preferably has an average molecular weight M_{W} in the range from 2000 to 40 000 g/mol, preferably 3,000 to 10,000 g/mol.

It is also possible to use copolymers of at least one monomer from the group consisting of monoethylenically unsaturated C₃-C₁₀-mono- or C₄-C₁₀-dicarboxylic acids or anhydrides thereof, such as maleic acid, maleic anhydride, acrylic acid, methacrylic acid, fumaric acid, itaconic acid and citraconic acid, with at least one hydrophilic or hydrophobic monomer as listed below.

Suitable hydrophobic monomers are, for example, isobutene, diisobutene, butene, pentene, hexene and styrene, olefins with 10 or more carbon atoms or mixtures thereof, such as, for example, 1-decene, 1-dodecene, 1-tetradecene, 1-hexadecene, 1-octadecene, 1-eicosene, 1-docosene, 1-tetracosene and 1-hexacosene, C₂₂-α-olefin, a mixture of C₂₀-C₂₄-α-olefins and polyisobutene having on average 12 to 100 carbon atoms per molecule.

Suitable hydrophilic monomers are monomers with sulfonate or phosphonate groups, and also nonionic monomers with hydroxyl function or alkylene oxide groups. By way of example, mention may be made of: allyl alcohol, isoprenol, methoxypolyethylene glycol (meth)acrylate, methoxypolypropylene glycol (meth)acrylate, methoxypolybutylene glycol (meth)acrylate, methoxy-poly(propylene oxide-co-ethylene oxide) (meth)acrylate, ethoxypolyethylene glycol (meth)acrylate, ethoxypolypropylene glycol (meth)acrylate, ethoxypolybutylene glycol (meth)acrylate and ethoxypoly(propylene oxide-co-ethylene oxide) (meth)acrylate. Polyalkylene glycols here may comprise 3 to 50, in particular 5 to 40 and especially 10 to 30 alkylene oxide units per molecule.

Particularly preferred sulfonic-acid-group-containing monomers here are 1-acrylamido-1-propanesulfonic acid, 2-acrylamido-2-propanesulfonic acid, 2-acrylamido-2-methylpropanesulfonic acid, 2-methacrylamido-2-methylpropanesulfonic acid, 3-methacrylamido-2-hydroxypropanesulfonic acid, allylsulfonic acid, methallylsulfonic acid, allyloxybenzenesulfonic acid, methallyloxybenzenesulfonic acid, 2-hydroxy-3-(2-propenyloxy)propanesulfonic acid, 2-methyl-2-propene-1-sulfonic acid, styrenesulfonic acid, vinylsulfonic acid, 3-sulfopropyl acrylate, 2-sulfoethyl methacrylate, 3-sulfopropyl methacrylate, sulfomethacrylamide, sulfomethylmethacrylamide, and salts of said acids, such as sodium, potassium or ammonium salts thereof.

Particularly preferred phosphonate-group-containing monomers are vinylphosphonic acid and its salts.

Moreover, amphoteric polymers can also be used as builders.

Inventive cleaning agents may comprise, for example, in the range from in total 10 to 50% by weight, preferably up to 20% by weight, of builder.

In one embodiment of the present invention, inventive cleaning agents according to the invention may comprise one or more co-builders.

Inventive cleaning agents may comprise one or more antifoams, selected for example from silicone oils and paraffin oils.

In one embodiment of the present invention, inventive cleaning agents comprise in total in the range from 0.05 to 0.5% by weight of antifoam.

Inventive cleaning agents may comprise one or more enzymes. Examples of enzymes are lipases, hydrolases, amylases, proteases, cellulases, esterases, pectinases, lactases and peroxidases.

In one embodiment of the present invention, inventive cleaning agents may comprise, for example, up to 5% by weight of enzyme, preference being given to 0.1 to 3% by weight. Said enzyme may be stabilized, for example with the sodium salt of at least one C₁-C₃-carboxylic acid or C₄-C₁₀-dicarboxylic acid. Preferred are formates, acetates, adipates, and succinates.

In one embodiment of the present invention, inventive cleaning agents may comprise at least one zinc salt. Zinc salts can be selected from water-soluble and water-insoluble zinc salts. In this connection, within the context of the present invention, water-insoluble is used to refer to those zinc salts which, in distilled water at 25° C., have a solubility of 0.1 g/l or less. Zinc salts which have a higher solubility in water are accordingly referred to within the context of the present invention as water-soluble zinc salts.

In one embodiment of the present invention, zinc salt is selected from zinc benzoate, zinc gluconate, zinc lactate, zinc formate, ZnCl₂, ZnSO₄, zinc acetate, zinc citrate, Zn(NO₃)₂, Zn(CH₃SO₃)₂ and zinc gallate, preferably ZnCl₂, ZnSO₄, zinc acetate, zinc citrate, Zn(NO₃)₂, Zn(CH₃SO₃)₂ and zinc gallate.

In another embodiment of the present invention, zinc salt is selected from ZnO, ZnO.aq, Zn(OH)₂ and ZnCO₃. Preference is given to ZnO.aq.

In one embodiment of the present invention, zinc salt is selected from zinc oxides with an average particle diameter (weight-average) in the range from 10 nm to 100 μm.

The cation in zinc salt can be present in complexed form, for example complexed with ammonia ligands or water ligands, and in particular be present in hydrated form. To simplify the notation, within the context of the present invention, ligands are generally omitted if they are water ligands.

Depending on how the pH value of mixture according to the invention is adjusted, zinc salt can change. Thus, it is for example possible to use zinc acetate or ZnCl₂ for preparing formulation according to the invention, but this converts at a pH of 8 or 9 in an aqueous environment to ZnO, Zn(OH)₂ or ZnO.aq, which can be present in non-complexed or in complexed form.

Zinc salt may be present in those inventive cleaning agents that are solid at room temperature. In such inventive cleaning agents zinc salts are preferably present in the form of particles which have for example an average diameter (number-average) in the range from 10 nm to 100 μm, preferably 100 nm to 5 μm, determined for example by X-ray scattering.

Zinc salt may be present in those inventive cleaning agents that are liquid at room temperature. In such inventive cleaning agents zinc salts are preferably present in dissolved or in solid or in colloidal form.

In one embodiment of the present invention, inventive cleaning agents comprise in total in the range from 0.05 to 0.4% by weight of zinc salt, based in each case on the solids content of the cleaning agent in question.

Here, the fraction of zinc salt is given as zinc or zinc ions. From this, it is possible to calculate the counterion fraction.

In one embodiment of the present invention, inventive cleaning agents are free from heavy metals apart from zinc compounds. Within the context of the present, this may be understood as meaning that inventive cleaning agents are free from those heavy metal compounds which do not act as bleach catalysts, in particular of compounds of iron and of bismuth. Within the context of the present invention, “free from” in connection with heavy metal compounds is to be understood as meaning that the content of heavy metal compounds which do not act as bleach catalysts is in sum in the range from 0 to 100 ppm, determined by the leach method and based on the solids content. Preferably, inventive cleaning agents has, apart from zinc, a heavy metal content below 0.05 ppm, based on the solids content of the formulation in question. The fraction of zinc is thus not included.

Within the context of the present invention, “heavy metals” are deemed to be all metals with a specific density of at least 6 g/cm³ with the exception of zinc. In particular, the heavy metals are metals such as bismuth, iron, copper, lead, tin, nickel, cadmium and chromium.

Preferably, inventive cleaning agents comprise no measurable fractions of bismuth compounds, for example less than 1 ppm.

Inventive cleaning agents are excellent for cleaning hard surfaces and fibres.

The invention is further illustrated by working examples.

The percentages of (A1) and (A2) as well as the percentage of crystalline versus amorphous salt (A) were determined by X-ray diffraction. The X-ray powder diffractometer measurements were carried out on a D8 Advance® diffractometer from Bruker AXS (Karlsruhe). In reflection with Cu—K α-radiation was measured with a variable diaphragm adjustment on the primary side and on the secondary side. The measurement range was 2° to 80° 2-theta, the step width 0.01° and the measurement time per angle step 3.6 seconds. Based on the software TOPAS from Bruker optics, the relative amounts of the two polymorphic forms of (A) were determined.

With exception of ee values and of degrees of crystallinity, percentages in the context of the examples refer to percent by weight unless expressly indicated otherwise.

Normal pressure: 1013 mbar. The abbreviation rpm stands for “revolutions per minute”.

Diaphragm is sometimes also referred to as membrane.

Average particle diameters are (D50) values and were determined by manual sieving methods unless expressly noted otherwise.

Salt (A.1): MGDA-Na₃ (racemic, determined by HPLC with penicillamine as chiral modification agent), provided as 40% by weight aqueous solution, pH: 13.

I. Manufacture of Inventive Particles of Salt (A.1) and Comparison Experiment

I.1 Manufacture of Inventive Particles of Salt (A.1)

Step (a.1): in step (a.1), 4.7 kg of a 40% by weight solution of salt (A.1) was filled into a crystallizer with stirrer and baffles. The solution was concentrated to 45% by weight by evaporation at 70° C. and 220 mbar. The solution was maintained at 70° C. under stirring with 500 rpm.

Step (b.1): under stirring, 12 g (0.63% of salt (A.1) in solution) of crystalline MGDA-Na₃ were added to the solution in the crystallizer. The resultant suspension was stirred for 20 minutes. Afterwards, the suspension of salt (A.1) was concentrated to 52.5% by weight by vacuum evaporation at 70° C. and 220 mbar in 4 hours. The suspension was maintained at 70° C. under stirring with 500 rpm for more than 3 hours.

Step (c.1): The resultant slurry was then filtered with a diaphragm filter press. The filtration pressure was raised from 0.5 bar and then to 2 bar. The resulting filter cake was then squeezed by a rubber diaphragm with a squeezing pressure of 8 bar for 8 minutes. A wet filter cake was obtained.

Step (d.1): The extrusion of the wet filter cake was performed in a “Brabender Messextruder” 19/20 DN manufactured by Brabender GmbH & Co.KG. The wet filter cake was blended with 10% by weight of dry MGDA-Na₃ powder manually to increase the viscosity. The temperature of the obtained paste in the extruder varied between 57° C. and 59° C. The pressure at the die with one hole of 2 mm varied between 50 und 58 bar. The rotational speed of the extruder screw was kept constant at 15 rpm. The rotational speed of the two cutter knives was kept constant at 100 rpm.

Step (e.1) and (f.1): The resultant wet particles from the extrusion, step (d.1), were dried in an oven, ground and classified in a fraction of particle size of from 0.2 mm to 1 mm, IP.1a, and of particle diameter <0.2 mm, IP.1b.

Inventive particles of salt (A.1) were obtained, IP.1a and IP.1b, respectively.

1.2 Manufacture of Comparative Particles of Salt (A.1)

The above protocol was repeated but step (d.1) was left out. Instead, the wet filter cake was dried in an oven and ground to powder. The drying was carried out in two steps: Vacuum drying 60° C., 12 hours, then grinding followed by final drying vacuum, 60° C., 12 hours. Comparative particles were obtained, C-IP.2.

II. Testing of Inventive Particles and of Comparative Particles

10 g of the respective inventive particles or comparative particles were mixed with 5 g Na-percarbonate and placed in a cell culture bottle with a semi permeable membrane to allow an exchange with the surrounding atmosphere. The vial was stored for 26 days in a climate-chamber at 35° C. and 70% humidity.

The discoloration—which is a yellowing in this case—of the stored mixtures was determined by measuring the b-value of the CIELAB color space (Elrepho measurement).

TABLE 1
Yellowing behavior

| | (P.1) | (P.2) | (P.3) | (P.4) | (P.5) |
|---|---|---|---|---|---|
| particles | [b-value] | [b-value] | [b-value] | [b-value] | [b-value] |
| IP.1a | 2.70 | 3.70 | 0.85 | −1.95 | 5.30 |
| IP.1b | 2.10 | 3.10 | 0.50 | −1.80 | 3.90 |
| C-IP.2 | 2.35 | 5.60 | 0.15 | 0.35 | 8.45 |

Peroxide Test:

(P.1): start value

(P.2): discoloration after storage for 12 days (delta to previous measurement)

(P.3): discoloration after storage for 19 days (delta to previous measurement)

(P.4): discoloration after storage for 26 days (delta to previous measurement)

(P.5): total discoloration

### III. EXAMPLE DETERGENT COMPOSITIONS

From inventive granules, example detergent compositions for automatic dishwashing detergents can be formulated by mixing the respective components according to Table 2.

TABLE 2
Example detergent compositions for automatic dishwashing

| All amounts in g/sample | ADW.1 | ADW.2 | ADW.3 |
|---|---|---|---|
| either of IP.1 or C-IP.2 | 30 | 22.5 | 15 |
| Protease | 2.5 | 2.5 | 2.5 |
| Amylase | 1 | 1 | 1 |
| n-C₁₈H₃₇—O(CH₂CH₂O)₉H | 5 | 5 | 5 |
| Sodium percarbonate | 10.5 | 10.5 | 10.5 |
| TAED | 4 | 4 | 4 |
| Na₂CO₃ | 19.5 | 19.5 | 19.5 |
| Sodium citrate dihydrate | 15 | 22.5 | 30 |
| HEDP | 0.5 | 0.5 | 0.5 |
| ethoxylated | optionally: | optionally: | optionally: |
| polyethylenimine, 20 | 0.1 | 0.1 | 0.1 |
| EO/NH group, | | | |
| Mₙ: 30,000 g/mol | | | |

The inventive detergent compositions furnish excellent automatic dishwashing results.

1. A process for making solid methylglycine diacetate (MGDA) alkali metal salt (A), said process comprising the steps of
(a) providing a 40 to 60% by weight aqueous solution of said salt (A) having a temperature in the range of from 50 to 90° C.,
(b) crystallizing salt (A),
(c) removing said crystalline salt (A) from the mother liquor by filtration, and
(d) subjecting the water containing filter cake from step (c) to an extrusion step.

2. The process according to claim 1, wherein in step (d), the extrusion step is carried out in a piston press, a twin screw extruder or a single-screw extruder.

3. The process according to claim 1, wherein step (d) is followed by at least one step selected from
(e) drying the extrudate, and
(f) grinding the extrudate.

4. The process according to claim 1, wherein salt (A) is selected from compounds according to general formula (I)
[CH3-CH(COO)—N(CH2-COO)2]M3-xHx (I)
wherein
M is selected from alkali metal cations, same or different, and
x is in the range of from zero to 1.0.

5. The process according to claim 1, wherein salt (A) is selected from the racemic mixture and mixtures of the enantiomers with predominantly the L-enantiomer with an ee value in the range of from 0.1 to 30%.

6. The process according to claim 1, wherein the filter cake from step (c) has a water content in the range of from 1 to 40% by weight.

7. The process according to claim 1, wherein such solid salt (A) has a residual moisture content in the range of from 0.5 to 12% by weight.

8. The process according to claim 1, wherein the pressure at the exit of the respective piston press, twin screw extruder or single-screw extruder is in the range of from 0.1 to 200 bar.

9. The process according to claim 1, wherein step (c) includes a diaphragm squeeze subsequently to filtration.

10. The process according to claim 1, wherein 5 to 20% of the product from step (e) or (f) is returned to the filter cake and subjected to step (d).

11. The process according to claim 1, wherein the aqueous solution provided in step (a) further comprises a (co)polymer of (meth)acrylic acid (B).

12. Column-shaped particles of methylglycine diacetate (MGDA) alkali metal salt (A) with an average diameter in the range of from 0.1 to 20 mm and a length of at least 1 mm, said particles comprising
(A1) in the range of from 1 to 10% by weight monoclinic alkali metal salt of MGDA, and
(A2) in the range of from 90 to 99% by weight orthorhombic alkali metal salt of MGDA, percentages referring to the entire content of the crystalline salt (A), and wherein the crystalline content is 80 to 99% by weight referring to the entire content of the respective salt (A).

13. The column-shaped particles according to claim 12, wherein salt (A) is selected from compounds according to general formula (I)
[CH3-CH(COO)—N(CH2-COO)2]M3-xHx (I)
wherein
M is selected from alkali metal cations, same or different, and
x is in the range of from zero to 1.0.

14. The column-shaped particles according to claim 12, wherein salt (A) is the trisodium salt.

15. A method for making laundry detergents or cleaners comprising combining the column-shaped particles of claim 12 with at least one bleaching agent.

16. The column-shaped particles according to claim 13, wherein salt (A) is the trisodium salt.

17. The method according to claim 15, wherein the at least one bleaching agent is at least one peroxy compound.
